(19) United States
(12) Patent Application Publication
(10) Pub. No.: US 2026/0144586 A1
(43) Pub. Date: May 28, 2026

(54) ADAPTIVE TISSUE TREATMENT

(71) Applicant: LUMENIS BE LTD., Yokneam (IL)

(72) Inventors: Bagrat Khachatryan, Yokneam (IL); Tal Grinberg, Yokneam (IL); Moshe Elazar, Yokneam (IL); Assaf Preiss, Yokneam (IL)

(21) Appl. No.: 19/356,505

(22) Filed: Oct. 13, 2025

Related U.S. Application Data
(60) Provisional application No. 63/706,929, filed on Oct. 14, 2024.

(51) Int. Cl. A61B 18/12; A61B 18/00

(52) U.S. Cl. CPC A61B 18/1206; A61B 2018/0047; A61B 2018/00577; A61B 2018/00589; A61B 2018/00648; A61B 2018/00702; A61B 2018/00791; A61B 2018/00875; A61B 2018/0091

(57) ABSTRACT

A device and system for aesthetic treatment of tissue, where the tissue may be, but not limited to skin and fat. The device or system comprises at least a processor, an RF generator and RF electrodes. The RF electrodes configured to be employed in monopolar, bipolar, multipolar or any combination of these modes and the electrodes further being configured to switch between these modes. The device and system configured to measure at least one efficacy parameter of the RF treatment before and during RF treatment and dynamically and in real time change the RF output power of the RF treatment based on that measurement.

### TECHNICAL FIELD

The present disclosure relates to the field of aesthetic devices and systems. Particularly, but not exclusively, the present disclosure relates to a system for radio frequency (RF) aesthetic treatment of different tissue types.

### BACKGROUND

Several RF energy treatments are available for different tissue types, such as skin and fat. Treatments using different modalities to deliver the RF energy treatment are also known. A few of many examples are micro-needling, a cannula, probe or handpiece with RF electrodes inside the body, or RF electrodes in a housing on the skin. Skin tightening and fat removal are two examples of RF treatments. Adipose tissue, more commonly known as “fat”, is formed of cells containing stored lipid. An excess of adipose tissue, i.e., obesity, may be unhealthful in that it gives rise to varying health problems in human beings both physical and psychological in nature.

### SUMMARY OF THE DISCLOSURE

Additional features and advantages are realized through the techniques of the present disclosure.

In one non-limiting embodiment of the disclosure, a device or system for aesthetic treatment of tissue, where the tissue may be, but not limited to skin and fat. The device or system comprises at least a processor, an RF generator and RF electrodes. The RF electrodes configured to be employed in monopolar, bipolar, multipolar or any combination of these modes. The electrodes may be configured to switch between these modes.

In another non-limiting embodiment, the device or system defines treatment parameters of the RF treatment and analyze the treatment outcome for a predicted treatment efficacy or clinical effect of those treatment parameters. Further, the processor, in some embodiments, changes or suggest changes to the treatment parameters and/or profiles to increase the predicted treatment efficacy. The changes may be suggested before and during the treatment. In some embodiments, a predicted efficacy look-up table is generated correlating the measured treatment parameters, such as impedance and temperature, which result in optimal treatment efficacy for each type of tissue treatment. The changes to treatment parameters may be done before and during RF treatment and dynamically and in real time change the output power of the RF treatment based on the measured treatment area parameters.

The foregoing summary is illustrative only and is not intended to be in any way limiting. In addition to the illustrative aspects, embodiments, and features described above, further aspects, embodiments, and features will become apparent by reference to the drawings and the following description.

### BRIEF DESCRIPTION OF THE ACCOMPANYING FIGURES

The above-mentioned aspects, other features and advantages of the disclosure will be better understood and will become more apparent by referring to the exemplary embodiments of the disclosure, as illustrated in the accompanying drawings.

FIG. 1 is a graph showing typical conductivity of various tissue types over a range of RF frequencies from 100 kHz to 6 MHz, according to some embodiments.

FIG. 2 is a graph showing typical behavior of electrical impedance of tissue as a function of temperature, according to some embodiments.

FIG. 3 is a functional block diagram of a device or system for tissue treatment, according to some embodiments.

FIG. 4 is a graph depicting behavior of RF output power of an RF generator, both unregulated and regulated, as a function of impedance, according to some embodiments.

FIGS. 5A-B depict a cannula of the treatment system, according to some embodiments.

FIG. 6 illustrates a cannula device of the treatment system, according to some embodiments.

FIG. 7 is a flowchart depicting steps in a method of tissue treatment, according to some embodiments.

FIG. 8 is a flowchart depicting steps in a method of tissue treatment, according to some embodiments.

The figures depict embodiments of the disclosure for purposes of illustration only. One skilled in the art will readily recognize from the following description that alternative embodiments of the unit illustrated herein may be employed without departing from the principles of the disclosure described herein.

### DETAILED DESCRIPTION

### Overview

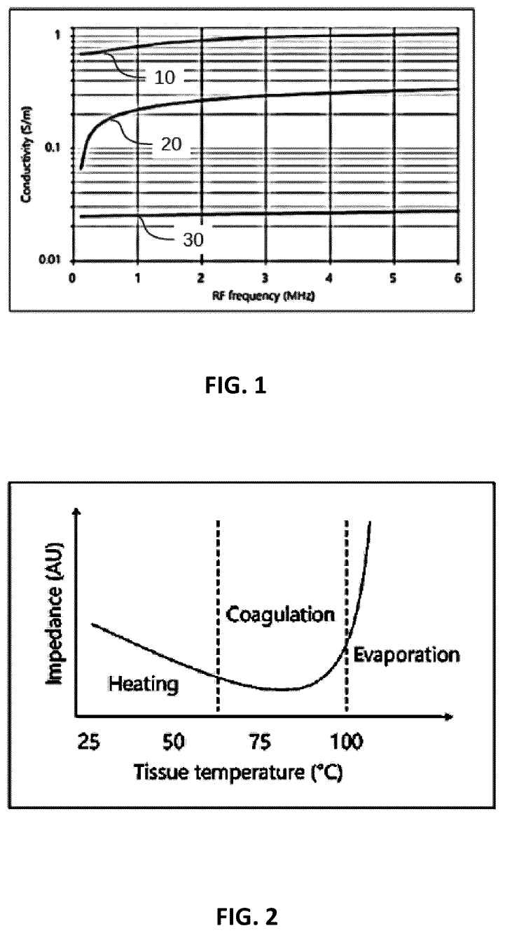

Reference is now made to FIG. 1, depicting behavior of conductivities of three kinds of bodily tissues over a range of RF frequencies of 100 kHz to 6 MHz. Shown are the behavior for blood 10, skin 20, and fat 30. Skin conductivity 20 has a gradient between 100 kHz and about 2 MHz with a lower and decreasing dependence on frequency between 3 and 6 MHz. Skin conductivity gradient is typically a higher value than the gradient of blood the fat. Fat tissue conductivity 30 is essentially independent of frequency throughout the range of 100 kHz to 6 MHz.

Reference is now made to FIG. 2, depicting behavior of electrical impedance of tissue as a function of temperature. The behavior may be categorized into three thermal regimes: a heating, a coagulation, and an evaporation (or ablation) regime.

In the heating regime, room temperature to about 65° C., warming of tissue reduces tissue impedance. This temperature dependence is related to several physical mechanisms, such as reduction in viscosity with increase in temperature.

In the coagulation regime, from about 65° C. to about 90° C., coagulation of tissue causes a chemical change in tissue structure, which affects the impedance behavior.

In the evaporation regime, where tissue is heat fed to between 90° C. and 100° C., evaporation of liquid in the tissue occurs, increasing tissue impedance substantially.

It is therefore an objective, in some embodiments, to measure electrical impedance of tissue during treatment, in order to determine the thermal regime or temperature of the tissue under treatment. Such objective and measurement are applicable to any treatment involving heating of tissue. However, the measurement is particularly advantageous during treatment with RF because, in some embodiments, the electrodes used to apply RF to tissue are nearby and/or the same electrodes used to measure impedance. This ensures that the tissue region being measured is the same as the tissue being RF treated.

Such determination of thermal regime or temperature may be used to make adjustments during treatment, as further described herein.

### Embodiments of a Tissue Treatment System

Some embodiments of the present disclosure will be described referring to the accompanying drawings. While some specific terms of “upper,” “lower,” “below”, “above”, “right,” or “left” and other terms containing these specific terms and directed to a specific direction will be used, the purpose of usage of these terms or words is merely to facilitate understanding of the present invention referring to the drawings. Accordingly, it should be noted that the meanings of these terms or words should not improperly limit the technical scope of the present invention.

The foregoing description broadly outlines the features and technical advantages of the present disclosure in order that the description of the disclosure that follows may be better understood. Additional features and advantages of the disclosure will be described hereinafter which form the subject of the disclosure. It should be appreciated by those skilled in the art that the conception and specific embodiments disclosed may be readily utilized as a basis for modifying or designing other devices for carrying out the same purposes of the present disclosure. It should also be realized by those skilled in the art that such equivalent constructions do not depart from the scope of the disclosure. It is to be expressly understood, however, that each of the figures is provided for the purpose of illustration and description only and is not intended as a definition of the limits of the present disclosure.

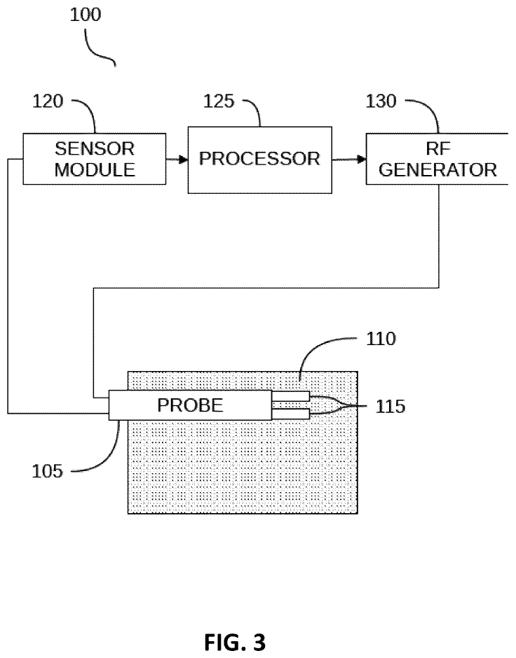

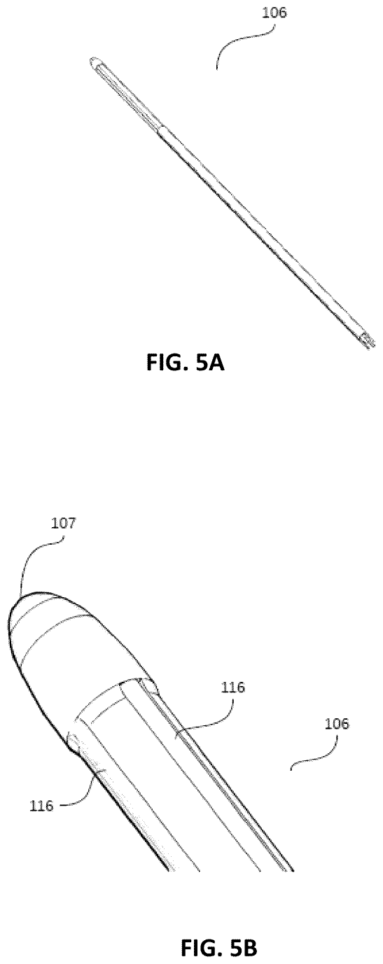

Reference is now made to FIG. 3, depicting, by way of specific example, a functional block diagram of a system 100 for RF aesthetic treatment, according to some embodiments.

The system 100 may comprise one or more RF electrodes 115. The RF electrodes 115 may be configured to be in contact with a tissue 110 of a subject.

In some embodiments, the RF electrodes 115 are disposed on a probe or handpiece 105 for delivery of treatment energy to a tissue. In some non-limiting embodiments, the probe or handpiece 105 is a cannula, inserted into the body of the subject. A cannula may be used where RF electrodes 115 are to contact sub-cutaneous tissues, such as adipose fat. In other embodiments, the handpiece 105 is non-invasive, whereby RF electrodes 115 are in contact with external skin. In some embodiments, the handpiece 105 is an RF micro-needling (RFMN) handpiece, whereby the RF electrodes 115 comprise an array of fine needles that contact and, in some cases, penetrate the skin. The handpiece 105 may be manually manipulated or may be motion controlled by a motion control mechanism.

The handpiece 105 may be inserted through a pre-established opening that reaches the tissue 110 or may pierce through the body until reaching the tissue 110.

The device 100 may comprise a sensor module 120. In some embodiments, the sensor module may include an impedance module (not shown) for determining electrical impedance (hereinafter, “impedance”). In some embodiments, the impedance module determines impedance measurements employing electrodes measuring voltage and current output to calculate the phase difference. In some embodiments and the sensor module measures temperature 120. In some embodiments, the sensor module 120 may be configured to make a measurement of one or more impedance or temperature of the tissue 110. In some embodiments, the sensor module 120 performs at least one measurement(s). In some embodiments, the sensor module 120, performs continual measurements at predetermined times during treatment.

In some embodiments, the sensor module 120 measures the impedance and/or temperature of tissue 110 in a region near the RF electrodes 115. In some embodiments, the sensor module 120 comprises two or more RF electrodes 115 dedicated to impedance measurement and an impedance measurement circuit. In such case, the sensor module 120 measures impedance of the portion of tissue 110 under treatment that is between the two or more RF electrodes 115 of the sensor module 120. In some embodiments, treatment RF electrodes 115 for delivering RF treatment may also be used for measuring impedance by the sensor module 120. In some embodiments, the sensor module 120 is a camera (not shown) that measures the treatment area and detects areas not to treat. The camera may also detect depth with a time of flight (TOF) ability. In some embodiments, the camera is also an IR sensor to measure temperature.

The device may comprise a processor 125. In this disclosure, “processor” (in the singular) is understood to mean one or more processors that may be hosted on a single computer or multiple computers, e.g. when features and functions of the processor are distributed over a plurality of networked computers.

The processor 125 receives the measurements/reading(s) generated by the sensor module 120. Based on the readings, the processor may determine impedance and thermal regime of the tissue. The tissue thermal regime may be one of heating, coagulation, and evaporation. Alternatively, or in addition, the tissue thermal regime is the temperature of the tissue 110.

In some embodiments, the processor 125 determines the tissue thermal regime based on a reading of tissue impedance. In some embodiments, sensor module 120 reads the tissue from the sensor module 120.

In some embodiments, the processor 125 determines the tissue impedance based on a reading of tissue temperature from the sensor module.

In some embodiments, the processor 125 receives tissue impedance and tissue temperature readings from the sensor module 120, using an impedance sensor and a temperature sensor. The processor 125 may be configured to improve the accuracy of either reading using output of other sensor. For example, and by way of specific example, if the impedance is measured through the electrodes 115 in contact with the tissue 110 and a temperature sensor measures temperature at a surface of the subject's skin, the processor 125 may improve on the temperature sensor reading by applying the impedance measurement readings, or a relative impedance change from room temperature, to determine the temperature at a point close to the electrodes 115 through which the impedance is measured. In another example, and by way of specific example, when in the coagulation regime, a temperature sensor disposed on an outer surface of the skin (further described herein) may be used to resolve on which side of the relative minimum of FIG. 2 is the temperature, which is then more locally (thus more accurately) determined by an impedance measurement reading.

The device may comprise an RF generator 130. The RF generator 130 generates one or more RF signals at the electrodes 115. The RF signals at the electrodes 115 are operative to heat tissue 110 surrounding the electrodes 115, for aesthetic treatment.

The RF generator 130 and the one or more electrodes 115 may be configured in a monopolar, bipolar, or multipolar configuration. For example, in a bipolar configuration, the processor 125 may cause RF from the RF generator 130 to be switched between any two RF electrodes 115 at a time. In a multipolar configuration, for example, the processor 125 may cause RF signal from the RF generator 130 to be divided into several parts, with each divided RF signal applied to a selected RF electrode 115.

In some embodiments, the configuration is adjustable between two or more of the configurations. In some embodiments, more than one configuration at the same time is present across different selected groups RF electrodes 115.

The RF generator 130 may generate RF signals according to a signal strength value received from the processor 125. The signal strength value may be a voltage, a current, a power, or any signal strength parameter to which the RF generator can respond. The sensor module 120, the processor 125, and the RF generator 130 may operate in a closed loop feedback, with the processor 125 configured to control the RF signal strength in response to conditions of the tissue 110, to optimize the treatment efficacy. In some embodiments, the closed loop feedback displays for a user of the treatment system a suggested change in RF signal strength in response to conditions of the tissue 110, to optimize the treatment efficacy. The user will then determine what changed to apply to the RF signal.

For example, in some embodiments, the processor 125 computes an RF signal strength value, based on the determined tissue impedance, so as to regulate a constant RF output power of the RF signal. In some embodiments, the tissue impedance is determined/measured over the time of the treatment, and therefore the RF output power may be adjusted over the time of the treatment.

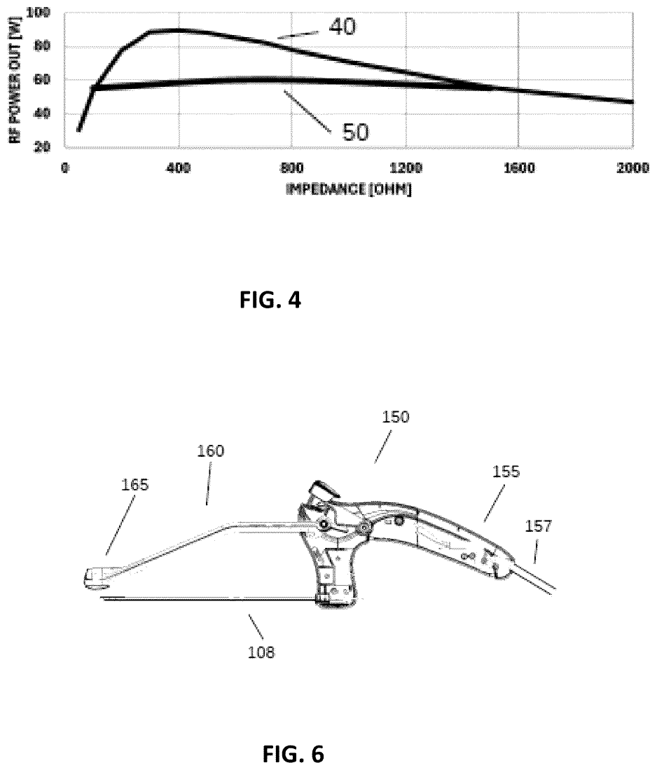

Reference is now also made to FIG. 4, a graph depicting RF output power of an RF tissue treatment device 100 as a function of impedance (ohms) of the tissue. Plot 40 represents the unregulated RF power output, showing that the RF output power is load dependent. The RF output power exceeds the level of power required to treat the tissue as seen by the impedance level. Plot 50 illustrates the regulated RF output power response, achieved by a processor 125 adjusting or stabilizing the RF output power. In some embodiments, the processor regulates the output power further employing an algorithm. Stabilization of the RF output power has many advantages; the power response of plot 50 may avoid burns and other side effects that are contrary to a successful treatment. In some embodiments, the impedance level is continuously measured, and the RF output power is changed dynamically and in real time, adjusted for effective treatment. Typically, there are multiple methods to measure impedance, including: a temperature sensor (given a known impedance vs. temperature behavior for the treated tissue), by direct measurement, for example, of voltage, current output and the phase difference between them from the RF electrodes 115, and with a dedicated impedance sensor.

In some embodiments, and by specific example, the processor 125 computes an RF signal strength value, based on the determined tissue temperature, so as to reach a selected thermal regime. When local thermal equilibrium is reached in the treated tissue, the processor may then compute an RF signal strength value so as to stabilize RF output power of the RF signal, as further described herein.

In some embodiments, and by specific example, the RF treatment system 100 may deliver RF energy while a temperature is below a predetermined safety level and if the predetermined temperature is unsafe the RF treatment system will discontinue the treatment.

In some embodiments, there are two additional electrodes (not shown) to measure impedance or existing electrodes may be configured to switch between treatment and measurements. Alternatively, or in addition, impedance may be measured based on the load on the RF generator 130. Thus, RF generation and impedance measurement may be simultaneous and with the same RF electrodes 115.

Typically, RF treatment devices or systems operate at predefined profiles and parameters such as a target or average RF output power over the treatment time or a threshold (minimum) tissue temperature. In some embodiments, there is a treatment optimization method of the treatment system 100 for a type of tissue 110, entailing measurement of one or more treatment parameter(s) of treatment profile(s), and/or other tissue characteristics (e.g., tissue density, color, water density, homogeneity, etc.) These treatment parameter(s) may include any of RF output power, RF frequency, tissue temperature, tissue impedance, relative changes in temperature in temperature and/or impedance, measured by the treatment system 100 or by other means. In some embodiments, the other tissue characteristics are determined by a tissue characteristics module (not shown) in or connected to the treatment system 100.

Further, and in some embodiments, the processor 125 performs treatment optimization. The processor 125 may determines for a type of tissue, based on the measured treatment parameter(s), and the predicted treatment efficacy or clinical effect of those treatment parameters. Further, the processor, in some embodiments, changes or suggest changes to the measured treatment parameters and/or profiles to increase the predicted treatment efficacy. The changes may be suggested before and during the treatment. In some embodiments, an efficacy look-up table is generated correlating the measured treatment parameters, such as impedance and temperature, which result in optimal treatment efficacy for each type of tissue treatment.

In some embodiments, tissue impedance is adjusted during treatment optimization by any manner appropriate such as, but not limited to, adding hydration gel to a tissue. In some embodiments, the tissue will be warmed with RF energy to reduce impedance, and therefore measuring impedance changes in the tissue may be correlated to temperature changes in the tissue and be indicative of how well the tissue will respond to treatment. In some embodiments of the RF treatment system 100, the RF output power of the RF treatment system is adjusted in real time during treatment based on the treatment parameters compared to predicted treatment efficacy of those parameters in the efficacy look-up table.

The optimal treatment parameter profile typically will differ from one tissue type to another tissue type and from subject to subject, as the dependence of predicted treatment efficacy on treatment parameters may vary between different tissues and different inside a specific tissue and different locations and subjects. The RF output power and other treatment parameters may then be customized for each patient and tissue to be treated in each patient.

After construction of an efficacy look-up table, the tissue treatment system 100 may be used to proceed with a treatment, whereby the processor 125 fetches optimized treatment parameters and proceeds to cause the tissue treatment system 100 to operate at optimized treatment parameters. In some embodiments, as the probe or handpiece 105 is moved and different types of tissue 110 are being treated with RF, the optimized treatment parameters are modified during treatment for the tissue 110 presently being treated.

In some embodiments, the tissue treatment system 100 is part of a skin diagnostic and treatment system using machine learning models. In some embodiments, the skin diagnostic and treatment system analyzes captured multi-spectral images to automatically determine the treatment parameters of the treatment system 100, including a customized treatment profiles In some embodiments, the skin diagnostic and treatment system analyzes a set of multi-spectral images taken right after treatment with the treatment system 100 to determine new treatment parameters/profiles and to customize treatments.

Reference is now made to FIGS. 5A and 5B, depicting, by way of specific example, multiple RF electrodes 116 on a cannula 106.

The multiple RF electrodes 116 may be employed, for example, as bipolar RF by switching between any two RF electrodes 116 at a time. Furthermore, when a cannula 106 is the preferred delivery system (handpiece 105 of an RF treatment system 100), the placement of the cannula 106 inside the body of a patient may be done after an aperture in the skin has been made by another medical instrument. In some embodiments of the RF treatment system 100, there are multiple RF electrodes 116 on the cannula 106, which may be at different distance (not shown) from the tip 107, to enable treatment of different portions of the tissue without moving the cannula after placement of the cannula into the body. In some embodiments, the handpiece 105 contains a temperature sensor (not shown) near the handpiece tip 107 and/or under one of the electrodes 116 (FIG. 5B). In some embodiments, the canula is hollow to provide for removing the adipose tissue (liposuction) before or after applying an RF signal.

Reference is now made to FIG. 6, depicting, by way of specific example, a tissue treatment handpiece 150 for aesthetic treatment.

In an exemplary embodiment, the handpiece 150 includes a cannula 107, such as one further described herein. The handpiece 150 may further comprise a handle 155. The handle 155 is configured for an operator of the handpiece 150 to insert the cannula 107 within a subject's tissue 110, traverse the cannula 106 through the tissue 110, and remove the cannula 107 from the tissue 110. Enclosed in the handle 155 may be some or all components of a tissue treatment system 100: a processor 125, and RF generator 130, and other electronics such as those associated with the sensor module 120. A cable harness 157 may contain wires and/or cables connecting the handpiece 150 to components of the treatment system 100 not included in the handle 155, to an electrical power source, and/or to a computer.

The handpiece 150 may further comprise an external arm 160. The external arm 160 is configured to be disposed outside of the subject's body during treatment. The external arm 160 may be pivotable about an axis on the handle 155, so as to allow the cannula 108 to traverse tissues at different depths from the subject's skin. On the external arm 160 may be a sensor head 165. The sensor head 165 may comprise one or more sensors such as a temperature sensor (e.g., a temperature sensor further described herein) and/or a camera. The external arm 160 may be spring loaded, so that the sensor head 165 maintains contact with the skin. The sensor head 160 may be disposed opposite the treatment tip of the cannula 108 (i.e., opposite the same place as the tissue under RF treatment). The position of the sensor head 165 on the skin surface can thereby indicate to an operator of the handpiece 150 at what position the cannula treatment tip is located. In some embodiments, a temperature or ultrasound sensor is used to detect the depth of the canula inside of the tissue.

### Embodiments of a Tissue Treatment Method

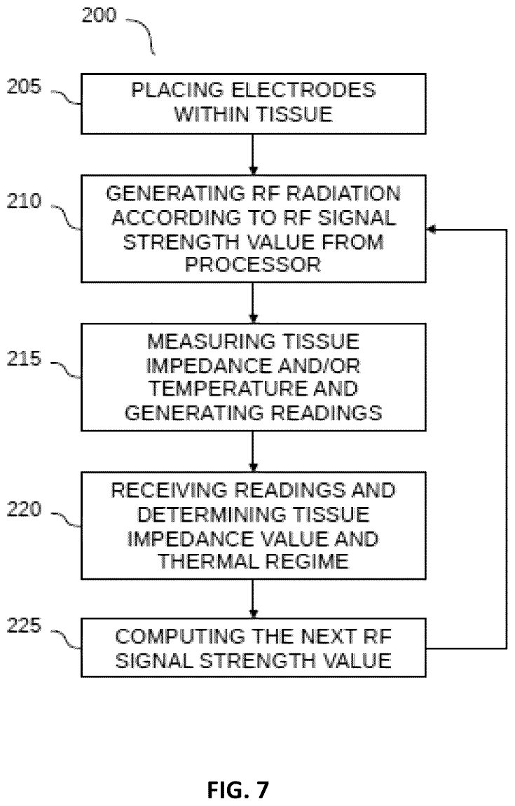

Reference is now made to FIG. 7, a flow chart depicting a method 200 of tissue treatment, according to some embodiments.

In an exemplary embodiment, and by way of specific example, the method 200 comprises placing one or more electrodes of an aesthetic treatment system in contact with a tissue of a subject 205. The tissue may comprise the epidermis, the dermis, and/or adipose tissue.

The method 200 may comprise generating RF signal at the electrodes 210. If the treatment is bipolar or multipolar, the RF signal may be generated across two or more of the electrodes. If the treatment is monopolar, the RF signal may be generated between an electrode in contact with the tissue and a return electrode attached to a skin surface of the subject. The return electrode may be large in area, relative the cross-sectional area of the tissue under treatment, and at a relatively far distance from the tissue under treatment. In some embodiments, the method at 210 is to generate a required amplitude of the RF current according to a strength value received from the processor.

The RF signal may be generated in at an RF signal strength value determined by a processor. At the start of treatment, the RF signal strength value may be some standard value. In some embodiments, the start of treatment, is based on the initial measurement of the tissue prior the RF treatment signal. In later iterations, the RF signal strength may be adjusted according to the treatment plan and the present conditions of the treatment.

To determine an appropriate value or range of values of the RF signal strength, the processor may utilize outputs from one or more sensors of the system. An impedance measurement reading and/or temperature sensor reading may at least one of tissue impedance and tissue temperature and generate reading(s) thereof 215.

The processor receives the readings and determines a tissue impedance value and a thermal regime of the tissue 220. The determined thermal regime may be heating, coagulation, or evaporation of the tissue. Alternatively, or in addition, the thermal regime may comprise the temperature itself. The temperature may be either as measured by the temperature sensor or determined from the measured impedance based, for example, on the tissue type. In some embodiments, the determined impedance value may be either one measured by measurements of the output voltage, current and the phase difference or determined from the temperature thermal regime based, for example, on the tissue type.

The processor, based on one or more of the impedance, thermal regime, and present RF signal strength, may compute an RF signal strength for the next iteration. The computation may be based on, for example, reaching a particular temperature or thermal regime and/or a desired rate of temperature change. When local thermal equilibrium is reached, the processor may compute an RF signal strength that will maintain a desired RF output power level, thereby stabilizing the RF output power level during the course of a treatment (or segment thereof) as further described herein. The desired RF output power level itself may be based on the treatment plan and present conditions of the tissue, as determined by the aesthetic treatment system, other system(s) used in connection with the treatment, a database, inputs by medical personnel conducting the treatment, or any combination thereof.

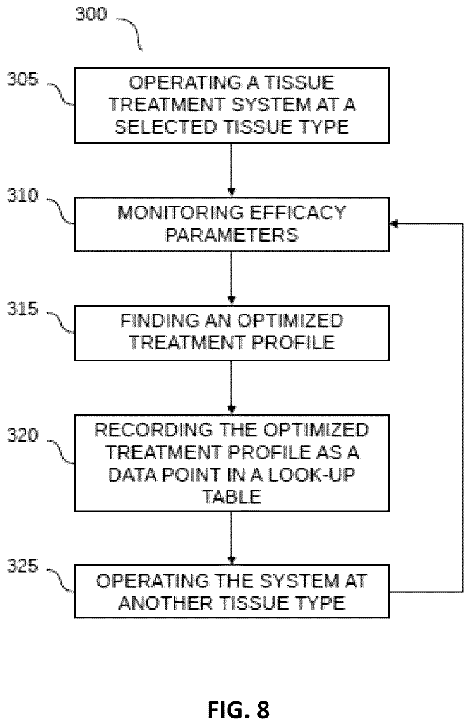

Reference is now made to FIG. 8, a flow chart depicting a method 300 of tissue treatment optimization. In some embodiments, the flow chart illustrates a training system for Artificial Intelligence for to develop a treatment model that learns optimal parameters of treatment from one type of tissue to apply to another.

In some embodiments, the method 300 comprises operating a tissue treatment system, such as further described herein, at a selected tissue type of a subject 305. Treatment parameters, such as RF power, RF frequency, and target temperature and impedance may be selected based on those of previous subjects or based on an estimate.

In some embodiments, the method 300 comprises monitoring efficacy parameters (predicted treatment efficacy predicted outcomes) during the treatment 310. Efficacy parameters may include extent of skin tightening, amount of fat removal, recoloration, etc.

In some embodiments, the method 300 comprises finding an optimized treatment profile of the treatment parameters 315. For example, a treatment may be performed at one part of the tissue type at 50 W of RF output power and at another part of the tissue type at 60 W. Other treatment parameters, such as RF frequency and target temperature and impedance may also be optimized. The treatment parameters are compared to predicted treatment efficacy of the treatments until finding an optimal treatment profile.

In some embodiments, the optimum treatment profile is recorded in a look-up table 320.

In some embodiments, the method 300 comprises operating the tissue treatment system at another tissue type 325. Steps 310, 315, and 320 are repeated for each tissue type.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprises”, “comprising”, or any other variations thereof, are intended to cover a non-exclusive inclusion, such that a mechanism that comprises a list of components does not include only those components but may include other components not expressly listed or inherent to such mechanism. In other words, one or more elements in the device or mechanism preceded by “comprises a . . . ” does not, without more constraints, preclude the existence of other elements or additional elements in the mechanism.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

While various aspects and embodiments have been disclosed herein, other aspects and embodiments will be apparent to those skilled in the art. The various aspects and embodiments disclosed herein are for purposes of illustration and are not intended to be limiting.

1. A system for aesthetic treatment of tissue, comprising
a handpiece 105, comprising one or more RF electrodes 115 configured to be placed in contact with a tissue 110 of a subject;
a sensor module 120, configured to make a measurement of one or more of impedance and temperature of said tissue 110 and generate at least one reading based on said measurement; a processor 125, configured to receive said one or more readings and determine one or more of an optimal impedance and optimal temperature of said tissue as a function of at least said one or more readings; and
an RF generator 130, configured to generate one or more RF signals for the one or more RF electrodes 115, with a strength of said RF signals according to an RF signal strength value received from said processor 125,
wherein said processor 125 is further configured to compute said RF signal strength value as a function of at least said measured or determined impedance reading, said determined temperature reading, or any combination thereof.

2. A system according to claim 1, wherein the sensor module further comprises an impedance module, configured to determine one or more of an impedance measurement of the tissue and generate at least one impedance reading, wherein determination of impedance is done by at least one of:
calculating a measurement of impedance; or
sensing a measure of impedance.

3. A system according to claim 1, wherein said processor is configured to compute said RF signal strength value over time of the treatment of tissue so as to regulate a constant RF output power of said RF signal.

4. A system according to claim 1, wherein the treatment of the tissue is a thermal regime, and the thermal regime comprises at least one of the following values:
heating of the tissue,
coagulation of the tissue, or
evaporation of the tissue.

5. A system according to claim 4, wherein said processor is configured to compute said RF signal strength value so as to reach a selected said thermal regime, and then after a local thermal equilibrium is reached so as to regulate a constant RF output power.

6. A system according to claim 1, wherein said handpiece comprises:
a cannula configured to be inserted into the body of the subject;
a non-invasive handpiece; or
a micro-needling handpiece.

7. A system according to claim 1, wherein said impedance measurement is made in a region of said one or more RF electrodes 115.

8. A system according to claim 7, wherein said processor is configured to determine the temperature as a function of said impedance reading.

9. A system according to claim 1, wherein said impedance module comprises at least two of said RF electrodes and an impedance measurement circuit configured to measure impedance of tissue between said at least two electrodes.

10. A system according to claim 9, wherein said at least two RF electrodes across which impedance is measured and said one or more RF electrodes across which an RF signal is generated constitute, at least in part, the same electrodes.

11. A system according to claim 10, wherein said impedance module measures impedance during generation of said one or more RF signals.

12. A system according to claim 1, wherein said sensor module comprises a temperature sensor.

13. A method for aesthetic treatment of tissue, comprising
providing a system for aesthetic treatment of tissue according to claim 1;
placing one or more RF electrodes of the handpiece in contact with a tissue of a subject;
measuring or determining one or more of impedance and temperature of said tissue to generate at least one reading thereof, by a sensor module of said device;
receiving said at least one reading and determining a treatment impedance value and treatment temperature value of said tissue as a function of at least said at least one reading, by a processor of said device;
computing said RF signal strength value, by said processor, as a function of at least said impedance value, said temperature, or any combination thereof; and
generating one or more RF signals, by an RF generator of said device, according to an RF signal strength value received from said processor.

14. A method according to claim 13, wherein said computing RF signal strength is made so as to regulate a constant RF output power over time of said RF signal.

15. A method according to claim 13, wherein the determined thermal regime comprises at least one of the following values:
heating of the tissue,
coagulation of the tissue, or
evaporation of the tissue.

16. A method according to claim 15, wherein said computing RF signal strength value is made so as to reach a selected said thermal regime, and then after a local thermal equilibrium is reached so as to regulate a constant RF output power.

17. A method according to claim 13, wherein said placing one or more RF electrodes is made by inserting a cannula into the body of the subject.

18. A method according to claim 13, wherein said determining of impedance is made in a region of said one or more RF electrodes.

19. A method according to claim 18, wherein said determining the treatment temperature is made as a function of said impedance reading.

20. A method according to claim 13, wherein said determining of impedance is made between at least two of the RF electrodes.

21. A method according to claim 20, wherein said determining of impedance is made during said generating of the RF signal.
